(19) United States
(12) Patent Application Publication
(10) Pub. No.: US 2016/0302791 A1
(43) Pub. Date: Oct. 20, 2016

(54) POWERED SURGICAL INSTRUMENT WITH A DEPLOYABLE ABLATION CATHETER

(71) Applicant: Covidien LP, Mansfield, MA (US)

(72) Inventor: JEFFREY SCHMITT, TRUMBULL, CT (US)

(21) Appl. No.: 14/689,639

(22) Filed: Apr. 17, 2015

(51) Int. Cl. A61B 17/10; A61B 17/068; A61B 18/00

(52) U.S. Cl. CPC A61B 17/105; A61B 18/00; A61B 17/068; A61B 2018/0091

(57) ABSTRACT

The present disclosure is directed to a surgical stapling apparatus. The surgical stapling apparatus includes a handle assembly, an elongated body extending distally from the handle assembly, and an end effector coupled to a distal end of the elongated body. The surgical stapling apparatus also includes an ablation catheter that extends through the handle assembly, the elongated body, and the end effector.

### BACKGROUND

1. Technical Field

The present disclosure relates to electromechanical surgical instruments, devices and/or systems for performing minimally invasive surgical procedures and methods of use thereof. More specifically, the present disclosure relates to an endoscopic surgical stapling apparatus that includes a deployable ablation catheter.

2. Description of Related Art

Surgical fastening devices, such as surgical stapling apparatuses, typically include two elongated jaw members to capture or clamp tissue. One jaw member carries a staple cartridge that houses a plurality of staples arranged in at least two lateral rows while the other jaw member has an anvil that defines a surface for forming the staple legs as the staples are driven from the staple cartridge. For the most part, the stapling operation is effected by cam members that travel longitudinally through the staples cartridge, with the cam members actuating upon staple pushers to sequentially eject the staples from the staple cartridge. A knife may travel between the staple rows to longitudinally cut or open the stapled tissue between the rows of staples. Examples of this kind of instrument are disclosed in U.S. Pat. Nos. 8,424,737 and 8,794,496.

Surgical stapling apparatuses may be used to perform a resection on an organ containing a lesion or tumor. Sometimes, an organ may have a secondary lesion at a separate location that is not within the surgically resectioned tissue. In order to treat the secondary lesion, the clinician has to withdraw the surgical stapling apparatus and permit an interventional radiologist or pulmonologist to perform an ablation procedure on the secondary lesion.

### SUMMARY

A surgical stapling apparatus is provided in embodiments of the present disclosure. The surgical stapling apparatus includes a handle assembly, an elongated body extending distally from the handle assembly, and an end effector coupled to a distal end of the elongated body. The surgical stapling apparatus also includes an ablation catheter that extends through the handle assembly, the elongated body, and the end effector.

A surgical system is also provided in embodiments of the present disclosure. The surgical system includes a surgical stapling apparatus having a handle assembly, an elongated body extending distally from the handle assembly, an end effector coupled to a distal end of the elongated body, and an ablation catheter that extends through the handle assembly, the elongated body, and the end effector. The system also includes a generator electrically coupled to the ablation catheter. The generator is configured to provide microwave energy to the ablation catheter.

In some embodiments, the handle assembly includes a control handle. A proximal end of the control handle may be coupled to the generator and a distal end of the control handle is coupled to the ablation catheter. Movement of the control handle causes movement of the ablation catheter. Specifically, the control handle includes a shaft that defines a longitudinal axis, wherein movement of the shaft along the longitudinal axis causes the ablation catheter to move along the longitudinal axis. Further, the control handle includes a rotation knob, wherein rotation of the rotation knob causes rotation of the ablation catheter.

In embodiments, the ablation catheter includes a coaxial cable couplable, at its proximal end, to a generator and at its distal end to a distal radiating section, the coaxial cable including inner and outer conductors and a dielectric positioned therebetween, the inner conductor extending distally past the outer conductor and in sealed engagement with the distal radiating section. The ablation catheter may also include a balun formed in part from a conductive material electrically connected to the outer conductor of the coaxial cable and extending along at least a portion of the coaxial cable.

In some embodiments, at least a portion of the outer conductor of the ablation catheter is removed to form a feedgap between the distal radiating section and the balun. Additionally, in embodiments a distal end of the ablation catheter is curved.

In other embodiments the end effector includes an anvil assembly and a cartridge assembly. The cartridge assembly includes an aperture configured to permit the ablation catheter to extend distally from the cartridge assembly or retract within the cartridge assembly.

In another embodiment, the surgical stapling apparatus includes a handle assembly, an elongated body extending distally from the handle assembly, and an end effector coupled to a distal end of the elongated body. The surgical stapling apparatus also includes an ablation catheter that extends through the handle assembly and exits near a distal end of the elongated body.

In yet another embodiment, the surgical stapling apparatus includes a handle assembly, an elongated body extending distally from the handle assembly, and an end effector coupled to a distal end of the elongated body. The apparatus also includes a port couplable to an electrosurgical generator. A conductor couples the port to a patch antenna or electrode disposed on the end effector.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a perspective view of a surgical stapling instrument according to embodiments of the present disclosure;

FIG. 2A is a top view of the surgical stapling instrument of FIG. 1;

FIG. 2B is a side view of the surgical stapling instrument of FIG. 1;

FIG. 3 is a cross-sectional view of a DLU and end effector of FIG. 1;

FIG. 4 is a perspective view of a microwave ablation catheter configured for use with the surgical stapling instrument shown in FIG. 1;

FIG. 5 is a system block diagram of the surgical stapling instrument according to embodiments of the present disclosure;

FIG. 6 is a side view of a resectioned organ having a secondary lesion that may be treated by an apparatus according to embodiments of the present disclosure;

FIG. 7 is a perspective view of a surgical stapling instrument according to another embodiment of the present disclosure;

FIG. 8 is a partial cross-sectional view of the surgical stapling instrument of FIG. 7;

FIG. 9 is a perspective view of a surgical stapling instrument according to another embodiment of the present disclosure; and

FIG. 10 is a partial cross-sectional view of the surgical stapling instrument of FIG. 9.

### DETAILED DESCRIPTION OF EMBODIMENTS

Embodiments of the presently disclosed electromechanical surgical system, instrument and/or device are described in detail with reference to the drawings, in which like reference numerals designate identical or corresponding elements in each of the several views.

This description may use the phrases “in an embodiment,” “in embodiments,” “in some embodiments,” or “in other embodiments,” which may each refer to one or more of the same or different embodiments in accordance with the present disclosure. For the purposes of this description, a phrase in the form “A or B” means “(A), (B), or (A and B)”. For the purposes of this description, a phrase in the form “at least one of A, B, or C” means “(A), (B), (C), (A and B), (A and C), (B and C), or (A, B and C)”.

The term “clinician” refers to any medical professional (i.e., doctor, surgeon, nurse, or the like) performing a medical procedure involving the use of embodiments described herein. As shown in the drawings and described throughout the following description, as is traditional when referring to relative positioning on a surgical instrument, the term “proximal” or “trailing” refers to the end of the apparatus which is closer to the clinician and the term “distal” or “leading” refers to the end of the apparatus which is farther away from the clinician.

The present disclosure is directed to a hand-held surgical stapling apparatus that includes a microwave ablation catheter. During a surgical procedure, a clinician may perform a resection or any other surgical procedure using the surgical stapling apparatus. In addition, the clinician may treat secondary lesions using the ablation catheter by extending the catheter through a distal end of the surgical stapling apparatus.

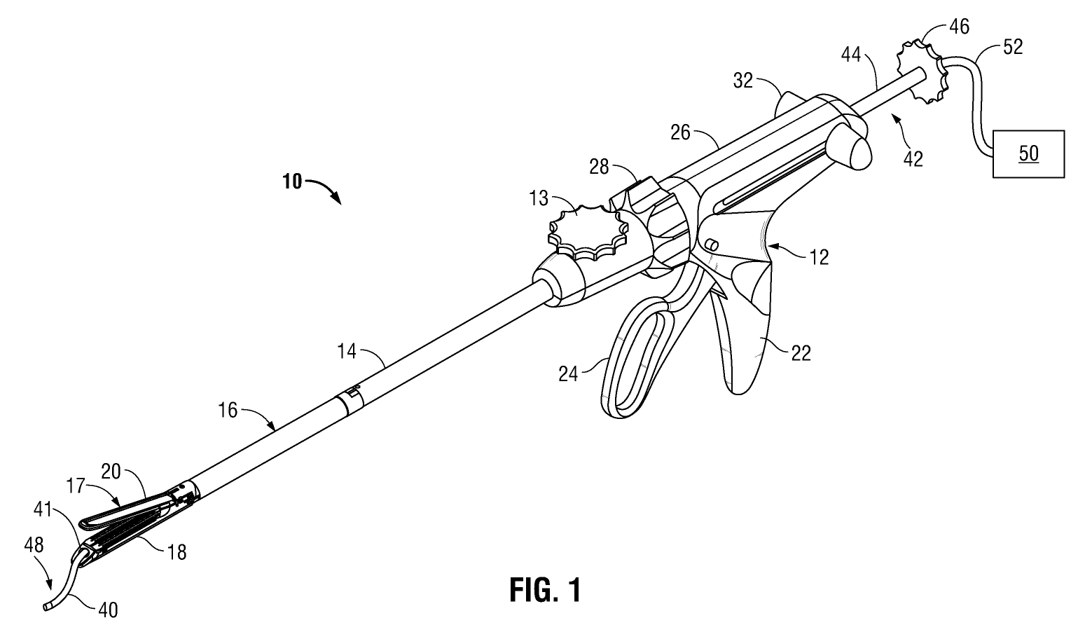

FIGS. 1-3 show a surgical stapling apparatus, generally referred to as 10. In the interest of brevity, this disclosure will focus primarily on systems, methods and structures for deploying an ablation catheter of surgical stapling apparatus 10. A detailed description of surgical stapling apparatus 10 may be found in U.S. Pat. No. 8,006,887, the entire disclosure of which is hereby incorporated by reference. Although the present disclosure is presented in the context of surgical stapling apparatus 10, the embodiments described herein are applicable to any apparatus having an articulating surgical tool.

Surgical stapling apparatus 10 is an endoscopic apparatus and includes a handle assembly 12 and an elongated body 14 extending therefrom. An end effector such as a loading unit which is replaceable and may be disposable, or DLU 16, is releasably secured to the distal end of elongated body 14. While the drawings illustrate a DLU 16, it is understood and within the scope of the present disclosure that a single use loading unit (SULU), a multi-use loading unit (MULU), or other end effector can equally be used in cooperation with surgical stapling apparatus 10. DLU 16 includes a tool assembly 17 having a cartridge assembly 18 housing a plurality of surgical staples and an anvil assembly 20 movably secured in relation to cartridge assembly 18. As seen in the FIG. 1, DLU 16 is configured to apply linear rows of staples. DLUs for applying any number of rows of staples, having staple pockets arranged in various patterns or DLUs and end effectors having various lengths, e.g., 30, 45 mm, or 60 mm, are also envisioned. U.S. Pat. No. 6,953,139, the disclosure of which is hereby incorporated by reference herein, includes a detailed discussion of various kinds of DLUs. A loading unit having various surgical end effectors may be used, including linear stapling tool assemblies. The linear stapling tool assemblies can include predetermined staple sizes and staple line lengths in various sizes and configurations. The stapling tool assemblies include circular, linear and other shapes.

Handle assembly 12 includes a stationary handle member 22, a movable handle member 24, and a barrel portion 26. A rotatable member 28 may be mounted on the distal end of barrel portion 26 to facilitate rotation of elongated body 14 with respect to handle assembly 12. Two retraction knobs 32 are movably positioned along barrel portion 26 to return surgical stapling apparatus 10 to a retracted position. An articulation knob 13 is mounted on the distal end of barrel portion 26 to facilitate articulation of tool assembly 17. The operation of the articulation knob 13 and its operative structure is set forth in detail in U.S. Pat. No. 7,481,348, the contents of which are hereby incorporated herein by reference.

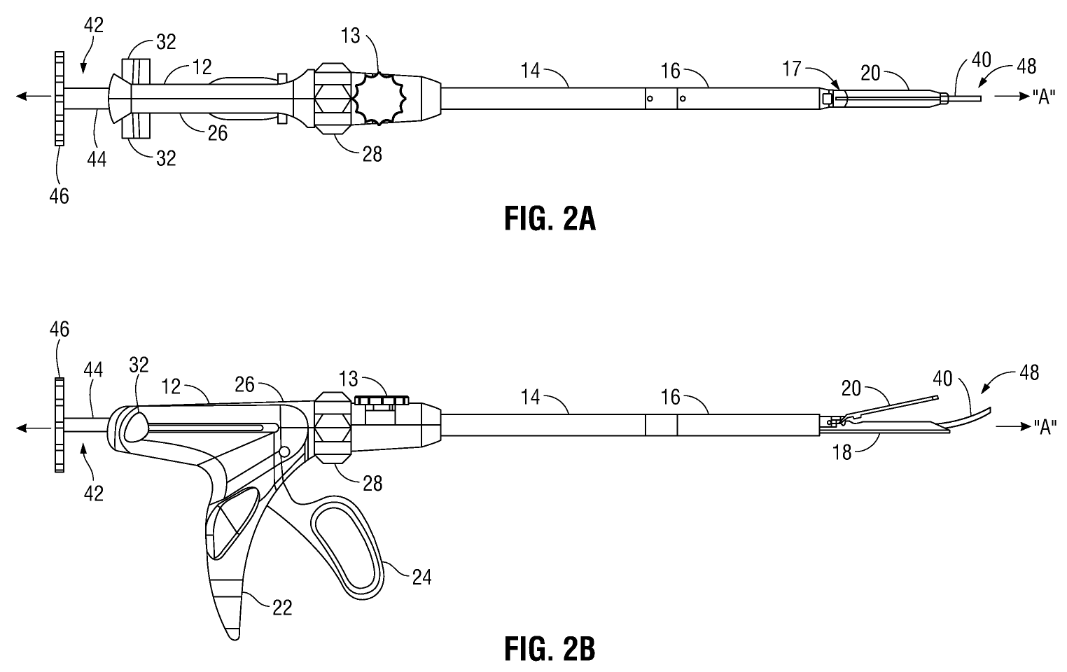

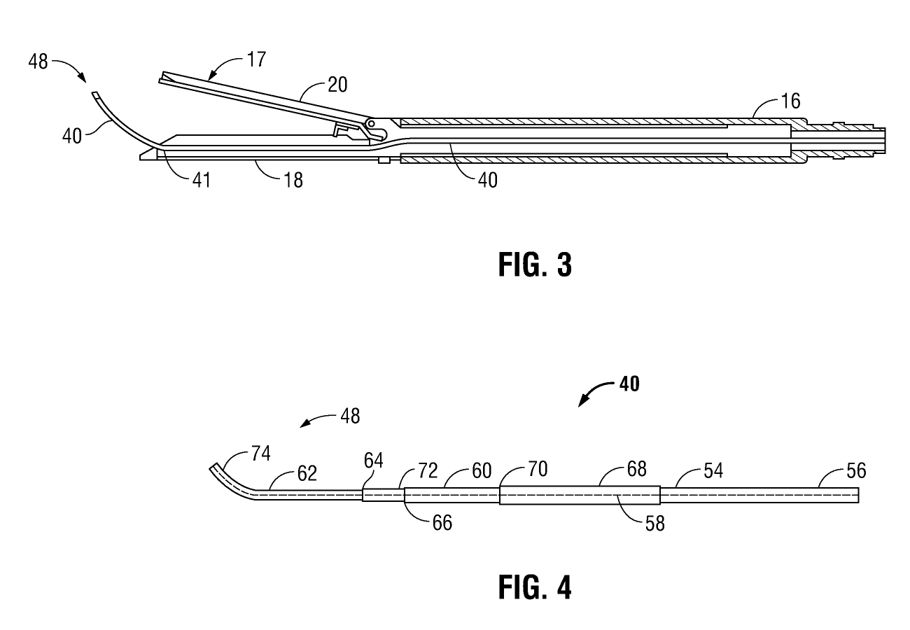

Surgical stapling apparatus 10 also includes a microwave ablation catheter 40 that extends through the handle assembly 12, elongated body 14 and DLU 16. Examples of microwave ablation catheters are set forth in detail in U.S. Patent Publication No. US 2014-0046315 A1, the entire contents of which are hereby incorporated by reference. The ablation catheter can extend distally from the DLU 16 through an aperture 41. A proximal end (not shown) of the ablation catheter 40 is connected to a distal end (not shown) of a control handle 42. The proximal end of the ablation catheter 40 and the distal end of the control handle 42 are connected via conventional means. Control handle 42 includes a shaft 44 that translates along longitudinal axis “A” defined by the shaft 44 as shown in FIGS. 2 and 3. The control handle also includes a rotation knob 46. Movement of the shaft 44 distally causes a distal end 48 of the ablation catheter 40 to advance distally while movement of the shaft proximally causes distal end 48 to retract within the DLU 16. Rotation knob 46 is used to rotate the ablation catheter 40 so that the distal end 48 may be oriented in a desired direction, such as toward a secondary lesion. The proximal end of the control handle 42 is electrically coupled to a generator 50 via a transmission line 52. Although FIGS. 1-3 show the ablation catheter 40 being housed in the cartridge assembly 18 of the DLU 16, the DLU 16 may be designed so that catheter 40 may be housed within the anvil assembly 20. Further, generator 50 may be incorporated into handle assembly 12.

Referring now to FIG. 4, the ablation catheter 40 is illustrated. Ablation catheter 40 includes a coaxial cable 54. Coaxial cable 54 includes a proximal end 56 that couples to the shaft 44 that provides electrical connection to an inner conductor 58 and outer conductor 60 of the coaxial cable 54 and the generator 50 (FIG. 1).

A distal radiating section 62 is provided at the distal end 64 of the coaxial cable 54 and is configured to receive the inner conductor 58. The distal radiating section 62 may be formed from any suitable material. In embodiments, the distal radiating section 62 may formed from ceramic or metal, e.g., copper, gold, silver, etc. The distal radiating section 62 may include any suitable configuration including but not limited to a blunt configuration, flat configuration, hemispherical configuration, pointed configuration, bar-bell configuration, tissue piercing configuration, etc. The distal radiating section 62 may couple to the distal end 64 of the coaxial cable via soldering, ultrasonic welding, adhesive, or the like. In one embodiment the distal radiating section 62 is sealed to the inner conductor 58 and a dielectric 66 to prevent fluid from contacting the inner conductor 58. As an alternative, the seal may be just between the inner conductor 58 and the dielectric 66.

An outer conductor 60 may be braided and extends along the dielectric 66 positioned between the inner and outer conductors 58, 60. As defined herein braided means made by intertwining three or more strands, and while described as a braid, the actual construction is not so limited and may include other formations of outer conductors of coaxial cables as would be understood by those of ordinary skill in the art. One advantage of a braided configuration of the outer conductor 60 is that it provides the ablation catheter 40 with some flexibility. Additionally, through the use of flat wire braiding and follow on braid compression with an appropriately sized die, the cross sectional dimension of the braided conductor may be minimized significantly in comparison to other conductive structures, such as a drawn copper tubing, while maintaining an acceptable electrical performance.

A choke or balun 68 is formed in part of a conductive layer (not shown) that extends along a portion of the coaxial cable 54. The conductive layer may be a braided material of similar construction as the outer conductor 60 and is connected to the outer conductor 60. Specifically, a portion of the outer conductor 60 is shorted (e.g., soldered, interbraided or otherwise affixed) to a proximal portion of the conductive layer (not shown).

The balun 68 also includes an insulative layer 70, which may be formed of polytetrafluoroethylene (PTFE). The insulative layer 70 is generally formed between the conductive material and the outer conductor 60. The insulative layer 70 extends distally past a distal end of the conductive material. The insulative layer 70 and its orientation extending beyond the conductive layer can be adjusted during manufacture to control the overall phase, energy field profile, and temperature response of the coaxial cable 54.

The outer conductor 60 extends distally beyond the insulative layer 70. A portion of the outer conductor 60 is removed to expose the dielectric 66 of the coaxial cable 54 and form a feedgap 72. The feedgap 72 is located distally from the balun 68 and proximal of and immediately adjacent the distal radiating section 62. The feedgap 72 and distal radiating section 62 are located and dimensioned to achieve a specific radiation pattern for the ablation catheter 40.

The distal end 48 of the ablation catheter 40 may have a curved end 74 that permits a clinician to aim the ablation catheter 40 in any desired direction, such as toward a secondary lesion as will be described below.

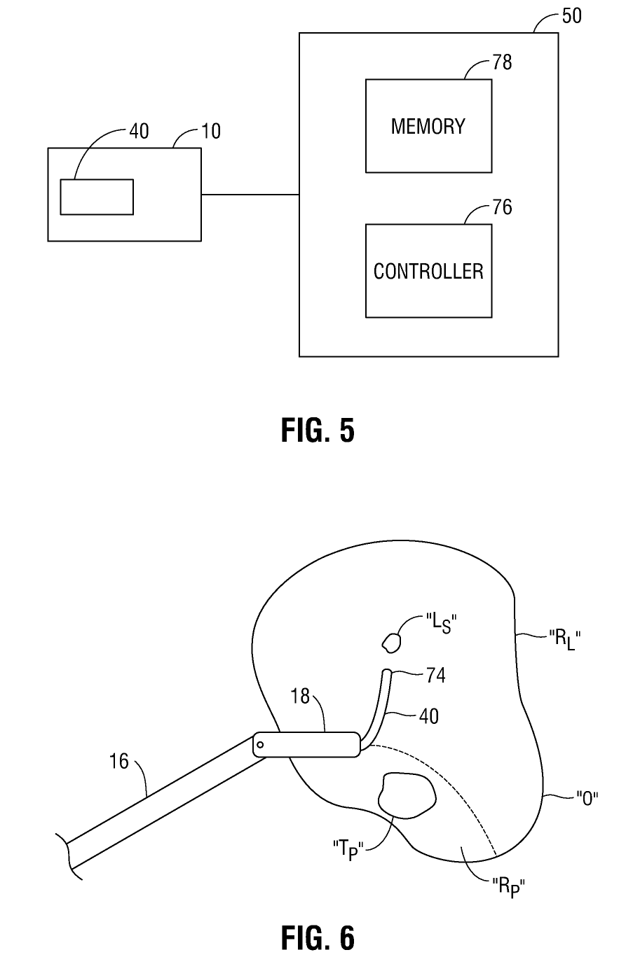

FIG. 5 depicts a system block diagram of the hand-held surgical instrument in accordance with an embodiment of the present disclosure. The ablation catheter 40 is electrically coupled via control handle 42 and transmission line 52 to generator 50. (See FIG. 1.) Generator 50 includes suitable input controls (e.g., buttons, activators, switches, touch screen, etc.) for controlling the generator 50. Examples of a generator 50 may be found in U.S. Pat. No. 8,797,039 and U.S. Publication No. US 2015-0025521 A1, the contents of which are hereby incorporated herein by reference. In addition, the generator 50 may include one or more display screens (not shown) for providing the user with variety of output information (e.g., intensity settings, treatment complete indicators, etc.). The controls allow the user to adjust power of the microwave energy, waveform, as well as the level of maximum energy allowed which varies depending on desired tissue effects and other parameters to achieve the desired waveform suitable for a particular task (e.g., coagulating, tissue sealing, intensity setting, etc.). The generator 50 may include a plurality of connectors to accommodate various types of instruments.

Generator 50 provides energy, such as microwave energy, to the ablation catheter 40. Microwave energy is energy having a wavelength in the range of 0.001-0.3 m. Generator 50 includes a controller 76 and a memory 78. Memory 78 executes an algorithm stored therein that when executed by the controller 76, causes the generator 50 to output microwave energy to the ablation catheter 40. Although not shown, ablation catheter 40 may include sensors that indicate whether the surgical procedure has been properly completed or whether the ablation catheter 40 is working properly or defective. The sensors would provide the information to the controller 76. The controller 76 may then alter the microwave energy, i.e., power, phase, duration, duty cycle, frequency, etc., being delivered or turn off the generator 50. Although generator 50 has been described above as an external generator, generator 50 may be incorporated into the handle 12 of the surgical stapling apparatus 10.

As shown in FIG. 6, during a surgical procedure, a clinician places the DLU 16 of the surgical stapling apparatus 10 within a patient. Using the DLU 16, the clinician may perform, e.g., a resection of an organ “O”. Before, during, or after the resection, the clinician may use the ablation catheter 40 to treat a secondary lesion “L_{S}” in a region “R_{L}” that is outside the region “R_{P}” to be resectioned, i.e., the region that includes primary tumor “T_{P}”. In order to treat the secondary lesion “L_{S}”, the clinician advances the control handle 42 distally causing the distal end 48 of the ablation catheter 40 to advance outward from the cartridge assembly 18. The clinician may then orient the curved end 74 toward the secondary lesion “L_{S}” by rotating the rotation knob 46 until curved end 74 is in close proximity of the secondary lesion “L_{S}”. The generator 50 is then activated in order to treat the secondary lesion “L_{S}”. Once treatment is complete and the generator 50 is turned off, the clinician then retracts the distal end 48 of the ablation catheter 40 within the cartridge assembly 18 by retracting the control handle 42 proximally.

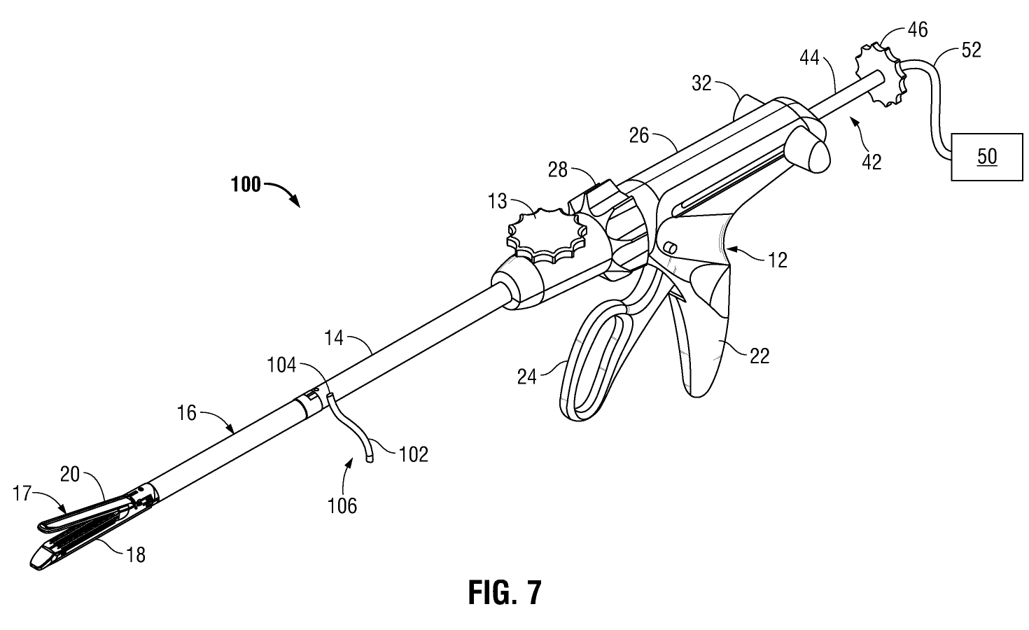

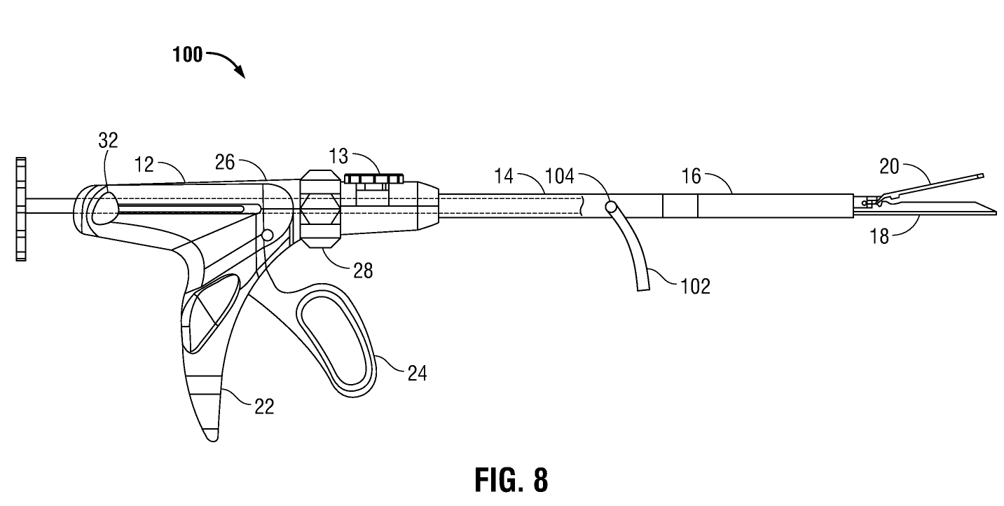

FIGS. 7 and 8 show a surgical stapling apparatus, generally referred to as 100, which is similar to surgical stapling apparatus 10 described above. In surgical stapling apparatus 100, the ablation catheter 102, which is substantially similar to ablation catheter 40, extends out of an aperture 104 in elongated body 14.

During a surgical procedure, a clinician places the DLU 16 of the surgical stapling apparatus 100 within a patient. Using the DLU 16, the clinician may perform, e.g., a resection of an organ. Before, during, or after the resection, the clinician may use the ablation catheter 102 to treat a secondary lesion in a region that is outside the region to be resectioned, i.e., the region that includes the primary tumor. In order to treat the secondary lesion, the clinician advances the control handle 42 distally causing the distal end 48 of the ablation catheter 102 to advance outward from the elongated body 14 through aperture 104. The clinician may then orient a curved end 106 of the ablation catheter 102 toward the secondary lesion by rotating the rotation knob 46 until the curved end 106 is in close proximity of the secondary lesion. The generator 50 is then activated in order to treat the secondary lesion. Once treatment is complete and the generator 50 is turned off, the clinician then retracts the distal end of the ablation catheter 102 within the elongated body 14 by retracting the control handle 42 proximally.

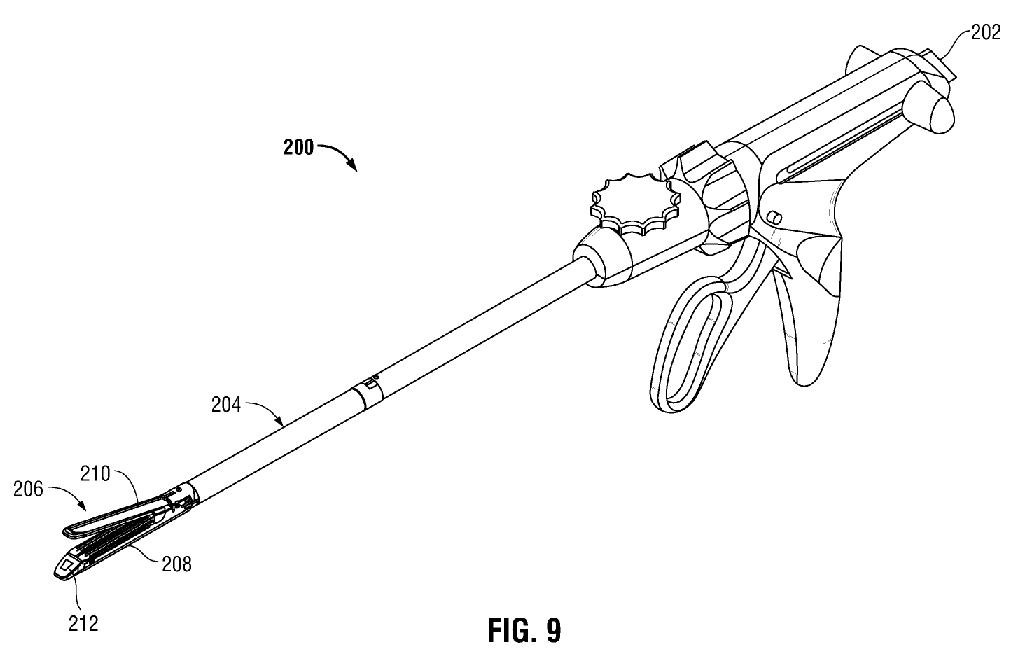

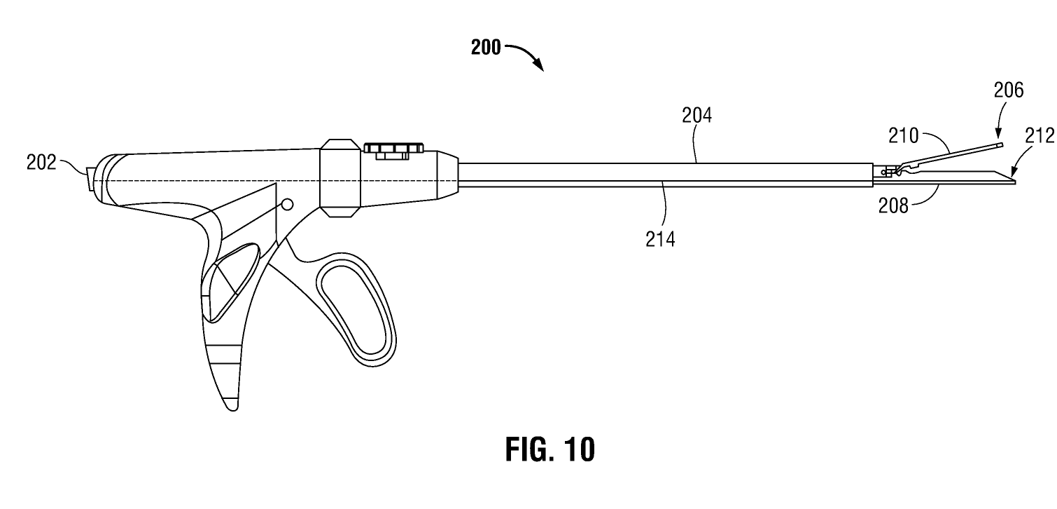

FIGS. 9 and 10 show a surgical stapling apparatus, generally referred to as 200, which is somewhat similar to surgical stapling apparatus 10 described above. Surgical stapling apparatus 200 includes a port 202 that may be coupled to an electrosurgical generator, e.g., generator 50, configured to provide microwave energy. A DLU 204 includes a tool assembly 206 having a cartridge assembly 208 housing a plurality of surgical staples and an anvil assembly 210 movably secured in relation to cartridge assembly 208. Cartridge assembly 208 includes an electrode 212 that is coupled to port 202 via a conductor 214. Electrode 212 may be a patch antenna that radiates microwave energy to perform an ablation procedure when tool assembly 206 is directed toward the secondary lesion. In other embodiments, port 202 may be coupled to an electrosurgical generator that provides radio frequency (RF) energy. Conductor 214 would provide the RF energy to electrode 212 in a monopolar arrangement.

During a surgical procedure, a clinician places the DLU 204 of the surgical stapling apparatus 200 within a patient. Using the DLU 204, the clinician may perform, e.g., a resection of an organ. Before or after the resection, the clinician may use the electrode 212 to treat a secondary lesion in a region that is outside the region to be resectioned, i.e., the region that includes the primary tumor. In order to treat the secondary lesion, the clinician orients the tool assembly 206 toward the secondary lesion until the electrode 212 is in close proximity of the secondary lesion. The generator (not shown) is then activated in order to treat the secondary lesion. Once treatment is complete and the generator is turned off, the clinician then proceeds to the resection procedure or removes the DLU 204 from the patient.

Although the above-described embodiments disclose an external electrosurgical generator, the electrosurgical generator may be incorporated into the handle of a surgical stapling apparatus.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, or the like. The controller may also include Field Programmable Gate Arrays (FPGA) and Complex Programmable Logic Devices (CPLD). The controller may also include a memory to store data and/or algorithms to perform a series of instructions.

Any of the herein described methods, programs, algorithms or codes may be converted to, or expressed in, a programming language or computer program. A “Programming Language” and “Computer Program” is any language used to specify instructions to a computer, and includes (but is not limited to) these languages and their derivatives: Assembler, Basic, Batch files, BCPL, C, C+, C++, Delphi, Fortran, Java, JavaScript, Machine code, operating system command languages, Pascal, Perl, PL1, scripting languages, Visual Basic, VHDL, Verilog, metalanguages which themselves specify programs, and all first, second, third, fourth, and fifth generation computer languages. Also included are database and other data schemas, and any other meta-languages. For the purposes of this definition, no distinction is made between languages which are interpreted, compiled, or use both compiled and interpreted approaches. For the purposes of this definition, no distinction is made between compiled and source versions of a program. Thus, reference to a program, where the programming language could exist in more than one state (such as source, compiled, object, or linked) is a reference to any and all such states. The definition also encompasses the actual instructions and the intent of those instructions.

Any of the herein described methods, programs, algorithms or codes may be contained on one or more machine-readable media or memory. The term “memory” may include a mechanism that provides (e.g., stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, optical signals, digital signals, and by other like signals.

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure.

What is claimed is:

1. A surgical stapling apparatus comprising:
a handle assembly;
an elongated body extending distally from the handle assembly;
an end effector coupled to a distal end of the elongated body; and
an ablation catheter that extends through the handle assembly, the elongated body, and the end effector.

2. The surgical stapling apparatus of claim 1, further comprising a control handle coupled to the ablation catheter.

3. The surgical stapling apparatus of claim 2, wherein movement of the control handle causes movement of the ablation catheter.

4. The surgical stapling apparatus of claim 2, wherein the control handle includes:
a shaft that defines a longitudinal axis, wherein movement of the shaft along the longitudinal axis causes the ablation catheter to move along the longitudinal axis; and
a rotation knob, wherein rotation of the rotation knob causes rotation of the ablation catheter.

5. The surgical stapling apparatus of claim 1, wherein the ablation catheter includes:
a coaxial cable couplable, at its proximal end, to a generator and at its distal end to a distal radiating section, the coaxial cable including inner and outer conductors and a dielectric positioned therebetween, the inner conductor extending distally past the outer conductor and in sealed engagement with the distal radiating section; and
a balun formed in part from a conductive material electrically connected to the outer conductor of the coaxial cable and extending along at least a portion of the coaxial cable.

6. The surgical stapling apparatus of claim 5, wherein at least a portion of the outer conductor of the ablation catheter is removed to form a feedgap between the distal radiating section and the balun.

7. The surgical stapling apparatus of claim 1, wherein a distal end of the ablation catheter is curved.

8. The surgical stapling apparatus of claim 1, wherein the end effector includes an anvil assembly and a cartridge assembly.

9. The surgical stapling apparatus of claim 8, wherein the cartridge assembly includes an aperture configured to permit the ablation catheter to extend distally from the cartridge assembly or retract within the cartridge assembly.

10. A surgical system comprising:
a surgical stapling apparatus including:
a handle assembly;
an elongated body extending distally from the handle assembly;
an end effector coupled to a distal end of the elongated body; and
an ablation catheter that extends through the handle assembly, the elongated body, and the end effector; and
a generator electrically coupled to the ablation catheter, the generator configured to provide energy to the ablation catheter.

11. The surgical system of claim 10, wherein the ablation catheter further comprises a control handle, wherein a proximal end of the control handle is coupled to the generator and a distal end of the control handle is coupled to the ablation catheter.

12. The surgical system of claim 11, wherein movement of the control handle causes movement of the ablation catheter.

13. The surgical system of claim 11, wherein the control handle includes:
a shaft that defines a longitudinal axis, wherein movement of the shaft along the longitudinal axis causes the ablation catheter to move along the longitudinal axis; and
a rotation knob, wherein rotation of the rotation knob causes rotation of the ablation catheter.

14. The surgical system of claim 10, wherein the ablation catheter includes:
a coaxial cable couplable, at its proximal end, to the generator and at its distal end to a distal radiating section, the coaxial cable including inner and outer conductors and a dielectric positioned therebetween, the inner conductor extending distally past the outer conductor and in sealed engagement with the distal radiating section; and
a balun formed in part from a conductive material electrically connected to the outer conductor of the coaxial cable and extending along at least a portion of the coaxial cable.

15. The surgical system of claim 14, wherein at least a portion of the outer conductor of the ablation catheter is removed to form a feedgap between the distal radiating section and the balun.

16. The surgical system of claim 10, wherein a distal end of the ablation catheter is curved.

17. The surgical system of claim 10, wherein the end effector includes an anvil assembly and a cartridge assembly.

18. The surgical system of claim 17, wherein the cartridge assembly includes an aperture configured to permit the ablation catheter to extend distally from the cartridge assembly or retract within the cartridge assembly.

19. A surgical stapling apparatus comprising:
a handle assembly;
an elongated body extending distally from the handle assembly;
an end effector coupled to a distal end of the elongated body; and
an ablation catheter that extends through the handle assembly and exits near a distal end of the elongated body.
